(19) United States
(12) Patent Application Publication
(10) Pub. No.: US 2023/0279345 A1
(43) Pub. Date: Sep. 7, 2023

(54) VASCULAR STRUCTURE-CONTAINING LARGE-SCALE BIOLOGICAL TISSUE AND CONSTRUCTION METHOD THEREOF

(71) Applicants: SHANDONG UNIVERSITY, Jinan (CN); YANSHAN UNIVERSITY, Qinhuangdao (CN)

(72) Inventors: Chuanzhen HUANG, Qinhuangdao (CN); Zhichao WANG, Jinan (CN); Hanlian LIU, Jinan (CN); Zhenyu SHI, Jinan (CN); Peng YAO, Jinan (CN); Dun LIU, Jinan (CN); Zhen WANG, Qinhuangdao (CN); Longhua XU, Qinhuangdao (CN); Shuiquan HUANG, Qinhuangdao (CN); Minting WANG, Qinhuangdao (CN); Hongtao ZHU, Jinan (CN); Bin ZOU, Jinan (CN)

(73) Assignees: SHANDONG UNIVERSITY, Jinan (CN); YANSHAN UNIVERSITY, Qinhuangdao (CN)

(21) Appl. No.: 17/851,731

(22) Filed: Jun. 28, 2022

(30) Foreign Application Priority Data
Mar. 1, 2022 (CN) 2022101980285

(51) Int. Cl. C12N 5/00; B33Y 10/00; B33Y 80/00; B29C 64/106

(52) U.S. Cl. CPC C12N 5/0068; B33Y 10/00; B33Y 80/00; B29C 64/106; C12N 2533/30; B29L 2031/7534

(57) ABSTRACT

A vascular structure-containing large-scale biological tissue and a construction method thereof. In the existing three-dimensional cell culture, it is contradictory for the elastic modulus of the scaffold material in ensuring structural stability and biocompatibility, and the vascular structure is required to provide channels for nutrient exchange when a large-scale structure is prepared. A cell-laden matrix material is poured into a hollow scaffold serving as a supporting scaffold. The overall stability of the scaffold structure can be ensured by regulating the mechanical properties of the supporting scaffold, thereby resolving the contradiction in ensuring structural stability and biocompatibility for the mechanical properties of the scaffold material in the conventional three-dimensional cell culture. A coaxially printing outer material contains a thermosensitive material. The removal of the outer thermosensitive material can increase the porosity of the vascular walls, and further increase the diffusion in the hollow vascular ducts.

### TECHNICAL FIELD

The present invention relates to the technical field of biological tissue, and specifically, to a vascular structure-containing large-scale biological tissue and a construction method thereof.

### BACKGROUND

Information disclosed in the background section is merely for better understanding of the overall background of the present invention, and is not necessarily regarded as acknowledging or suggesting, in any form, that the information constitutes the prior art known to a person of ordinary skill in the art.

In a three-dimensional cell culture, to ensure the stability of a scaffold structure, the scaffold material requires a high elastic modulus. For in vitro culture, the mechanical properties of the scaffold material will affect the survival, proliferation and differentiation of cells. Therefore, to ensure that the scaffold has good biocompatibility, it is necessary to make the mechanical properties of the scaffold material as close as possible to those required for cells in the in vivo environment. However, it is different in the mechanical properties for different cells in the living environment in vivo, for example, a lower elastic modulus for brain cells and a higher elastic modulus for bone cells. Therefore, it is contradictory for the mechanical properties of the scaffold material in ensuring structural stability and biocompatibility.

In addition, the nutrients are limited in diffusion distance, so it is necessary to introduce vascular structures to ensure the long-term viability and function of the model in the preparation of large-scale tissues and organs. There are two main methods for preparing vascular structures in vitro: direct and indirect preparation thereof. The direct preparation mainly adopts coaxial extrusion to print out hollow ducts. In study of those skilled in the art, for coaxial extrusion, a sacrificial material is first printed as an inner portion and then is removed after the entire structure has been fabricated, to obtain a hollow structure. Coaxial printing can directly print out a hollow duct structure. The indirect preparation of vascular structures includes first preparation of a model with a sacrificial material, then wrapping the model in a hydrogel material, and then removal of the sacrificial material after the hydrogel material is cured, to prepare the vascular structures.

The inventor believes that a diffusivity in the existing vascular structures prepared in vitro will be affected by a porosity of the vascular walls. However, at present, the porosity of the walls of the vascular structures, whether directly or indirectly prepared, depends on the properties of the material itself, and has not been effectively regulated.

### SUMMARY

In view of the defects in the prior art, the present invention provides a vascular structure-containing large-scale biological tissue and a construction method thereof.

According to a first aspect of the present invention, a vascular structure-containing large-scale biological tissue is provided. The biological tissue is composed of a cell-laden hydrogel matrix and a supporting scaffold, where the supporting scaffold is a hollow duct inserted into the cell-laden hydrogel matrix and contains a thermosensitive material.

The design ideas for the large-scale biological tissue provided in the first aspect are as follows: The supporting scaffold, which can enhance the mechanical properties and ensure the stability of the structure, is similar to the rebar bracket in house construction. The cell-laden hydrogel matrix, which is poured on the supporting scaffold, is similar to concrete. The supporting scaffold can ensure the structural stability. Therefore, during the design process of the cell-laden hydrogel matrix, only its biological properties need to be considered, without need to balance the mechanical properties like the materials used in general 3D bioprinting technology. It can not only make biological properties of the materials be close to the in vivo tissues where the cells are located but also resolve the inconsistence or even contradiction between the structural stability and biocompatibility for the mechanical properties of the materials.

In addition, to ensure that the cells inside the tissue can acquire sufficient oxygen and nutrients, the supporting scaffold is designed as a hollow duct structure. During tissue culture, the hollow duct is able to transport nutrients and oxygen to the cells inside the tissue and transport the wastes of cell metabolism out, which can exert the physiological function of the vascular structure in biological tissue. To further improve the diffusion of nutrients in the supporting structure, a thermosensitive material is incorporated in the duct walls of the supporting scaffold. The thermosensitive material can undergo a reversible sol-gel transition with the change in temperature. By regulating the ambient temperature, the thermosensitive material in the supporting scaffold changes from a gel state to a liquid state and dissolves into the surrounding environment, thereby increasing the porosity of the duct walls of the supporting structure and improving the diffusion of nutrients in the vascular structure.

Further, according to a second aspect of the present invention, a method for constructing the vascular structure-containing large-scale biological tissue is provided. The method includes: coaxially printing inner and outer materials on a receiving platform to form a scaffold structure, the outer material being a composite material of a thermosensitive material and a crosslinking material, and the inner material being a thermosensitive material; curing the outer material of the scaffold structure after the printing to obtain a stable scaffold structure; incubating the stable scaffold structure at a melting temperature of the thermosensitive material to melt the thermosensitive material of the inner and outer materials to obtain a hollow supporting scaffold; and pouring a cell-laden hydrogel matrix into the supporting scaffold and curing it.

The beneficial effects of one or more of the above technical solutions are as follows:

In the present invention, a supporting scaffold is introduced to provide structural support for the prepared biological tissue, and a cell-laden matrix material is poured on the supporting scaffold. The overall stability of the scaffold structure can be ensured by regulating the mechanical properties of the supporting scaffold, and the cells can have an ideal living environment by regulating the mechanical properties of the matrix material. This method can effectively resolve the contradiction in ensuring structural stability and biocompatibility for the mechanical properties of the scaffold material in the conventional three-dimensional cell culture.

The supporting scaffold is prepared into a hollow structure. During tissue culture, the hollow vascular structure can transport nutrients and oxygen to the cells in the matrix material and transport out the metabolism wastes of the cells in the matrix material, which can resolve the problem of necrosis of internal cells due to the inability to acquire sufficient nutrients due to the excessive size of the tissue during the three-dimensional culture of cells in vitro. Therefore, a large-scale tissue structure can be prepared by this method.

A hollow supporting scaffold structure is prepared by coaxially printing. A thermosensitive material is added into the outer material and then removed from the outer composite material after the entire scaffold structure is completed, so that the porosity of the hollow duct walls can be regulated, and the diffusion in the hollow ducts can be further improved.

### BRIEF DESCRIPTION OF THE DRAWINGS

The accompanying drawings constituting a part of the present invention are used to provide a further understanding of the present invention. The exemplary examples of the present invention and descriptions thereof are used to explain the present invention, and do not constitute an improper limitation of the present invention.

FIG. 1 is a schematic diagram of the vascular structure-containing large-scale biological tissue prepared in Example 1,
where panel a is a schematic diagram of the supporting scaffold structure, and panel b is a schematic diagram of the entire structure of the biological tissue.

FIG. 2 shows the vascular structure-containing large-scale biological tissue and its preparation process in Example 1 with
a scale of 1 cm, where panel a shows the sterilized hollow supporting scaffold structure, panel b shows the hollow supporting scaffold structure after being soaked in the culture medium, and panel c shows the entire structure of the hollow supporting scaffold with the cell-laden hydrogel matrix poured.

FIG. 3 shows the characterization of living/dead cell staining with
a scale of 200 μm, where panel a shows the staining of the cells cultured for 1 day, and panel b shows the staining of the cells cultured for 7 days.

FIG. 4 shows cell survival and proliferation,
where panel a shows the characterization of cell viability, and panel b shows cell proliferation.

FIG. 5 shows the diffusion of small molecules in the hydrogel ducts.

### DETAILED DESCRIPTION

It should be pointed out that the following detailed descriptions are all illustrative and are intended to provide further descriptions of the present invention. Unless otherwise specified, all technical and scientific terms used herein have the same meanings as those usually understood by a person of ordinary skill in the art to which the present invention belongs.

It should be noted that the terms used herein are merely used for describing specific implementations, and are not intended to limit exemplary implementations of the present invention. As used herein, the singular form is also intended to include the plural form unless the context clearly dictates otherwise. In addition, it should further be understood that, terms “comprise” and/or “include” used in this specification indicate that there are features, steps, operations, devices, components, and/or combinations thereof.

As described in the background, in the existing 3D cell culture engineering, it is contradictory for the mechanical properties of the scaffold in ensuring structural stability and biocompatibility, and there is a lack of regulatory mechanisms for the permeability of vascular structures. To resolve the foregoing technical problems, the present invention provides a vascular structure-containing large-scale biological tissue and a construction method thereof.

According to a first aspect of the present invention, a vascular structure-containing large-scale biological tissue is provided. The biological tissue is composed of a cell-laden hydrogel matrix and a supporting scaffold, where the supporting scaffold is a hollow duct inserted into the cell-laden hydrogel matrix to mimic vascular structures and contains a thermosensitive material.

In the large-scale biological tissue according to the first aspect, the supporting scaffold can mimic the vascular structure in the biological tissue to provide nutrients for the culture of cells and tissues and discharge waste metabolites.

The supporting scaffold is one or more ducts connected or not connected to each other. In an implementation provided by the present invention, the supporting scaffold has multiple layers of ducts superposed in the cell-laden hydrogel matrix, and the ducts of adjacent layers are arranged crosswise or vertically.

The size of the biological tissue may be designed and prepared according to specific application requirements. In a feasible implementation of the present invention, the size of the biological tissue may be adjusted in the range of 16×16 mm-40×40 mm.

The diameter of the duct in the supporting scaffold is 0.31-1.28 mm.

To meet different requirements for biological tissue culture and achieve the adjustable permeability of vascular structure, the permeability of the supporting scaffold is adjustable by changing the concentration of the crosslinking material in the coaxially printing outer material.

According to a second aspect of the present invention, a method for constructing the vascular structure-containing large-scale biological tissue is provided. The method includes: coaxially printing inner and outer materials on a receiving platform to form a scaffold structure, the outer material being a composite material of a thermosensitive material and a crosslinking material, and the inner material being a thermosensitive material; curing the outer material of the scaffold structure after the printing to obtain a stable scaffold structure; incubating the stable scaffold structure at a melting temperature of the thermosensitive material to melt the thermosensitive material of the inner and outer materials to obtain a hollow supporting scaffold; and pouring a cell-laden hydrogel matrix into the supporting scaffold and curing it.

Preferably, the coaxially printing inner and outer materials on a receiving platform to form a scaffold structure includes:

preparing the inner and outer materials for coaxially printing, the inner material being the thermosensitive material, and the outer material being a composite material of the thermosensitive material and the crosslinking material, charging the liquid inner and outer materials into syringes separately, and incubating them at a first temperature to form a gel;

loading the syringes containing the gel-like inner and outer materials into an extrusion 3D printer, connecting a nozzle for coaxial printing to the syringe, setting process parameters of the 3D printer, activating the receiving platform of the 3D printer, and setting a temperature of the receiving platform; and
starting software of the 3D printer to enable the nozzle for coaxial printing to move according to a predetermined trajectory and extrude the gel materials from the syringe on the receiving platform to form the scaffold structure, and processing the scaffold structure to cure the crosslinking material in the outer composite material, to obtain the stable scaffold structure.

Further, in the printing method, the inner thermosensitive material is Pluronic F127 or its derivatives or gelatin; further, the inner thermosensitive material is Pluronic F127.

Further, the crosslinking material is polyethylene glycol diacrylate, gelatin methacrylate, sodium alginate, or a mixture thereof; specifically, the crosslinking material is polyethylene glycol diacrylate.

In an implementation of the foregoing preferred technical solutions, the outer gel material is a composite material of polyethylene glycol diacrylate and Pluronic F127.

In the step of printing the scaffold structure, the inner material is formulated by dissolving Pluronic F127 in a phosphate solution at a mass fraction of 10-30%; and the outer material is formulated by fully dissolving a photoinitiator and polyethylene glycol diacrylate in a phosphate solution, prior to addition of Pluronic F127 for dissolving, where Pluronic F127 has a mass concentration of 10-30% and polyethylene glycol diacrylate has a mass concentration of 5-20%.

Based on the foregoing formulation of the outer material, the outer material may be cured by UV light or ion crosslinking. Considering the convenience of operation, in a preferred solution, the outer material is cured by UV light.

Further, the first temperature is 4-40° C., and the incubation time is 10-30 min; specifically, the first temperature is set to 37° C., and the incubation time is 20 min.

Further, the outer specification of the nozzle for coaxial printing is 14-21 G, and the inner specification of the nozzle for coaxial printing is 18-30 G; specifically, the outer specification of the nozzle for coaxial printing is 17 G, and the inner specification of the nozzle for coaxial printing is 22 G.

Further, during 3D printing, a moving speed of the nozzle for coaxial printing is 400-800 mm/min, and an inner-to-outer extrusion speed ratio is 1:4-1:1.

Further, the receiving platform is a high-temperature platform or a low-temperature platform with a temperature set to 4-50° C.; in a specific implementation, the receiving platform is a high-temperature platform with a temperature set to 50° C.

According to the second aspect, during the construction of the hollow supporting scaffold, the stable scaffold structure is soaked in a phosphate buffer or ultrapure water for incubation for 1-5 days, with a melting temperature of the thermosensitive material being 4-40° C.

Further, the stable scaffold structure is soaked in ultrapure water for incubation at a temperature of 4° C. for 1-3 days.

Further, after the hollow supporting scaffold is constructed, the method also includes a step for sterilization by soaking the hollow supporting scaffold in a 75% ethanol solution for 1-5 h, specifically, for 3 h. After the foregoing step for sterilization is completed, the hollow supporting scaffold needs to be washed to avoid the effect of residual ethanol. The supporting scaffold may be washed with a phosphate buffer and then placed in a complete medium for use.

In the construction method according to the second aspect, the cell-laden hydrogel matrix material includes but is not limited to collagen, hyaluronic acid, sodium alginate, gelatin methacrylate, or a combination thereof, with a mass concentration of 5-15%; preferably, in some embodiments, the cell-laden hydrogel matrix material is gelatin methacrylate with a mass concentration of 5%.

Further, the cell-laden hydrogel matrix is cured by ion crosslinking or photocrosslinking; preferably, in some embodiments, by photocrosslinking.

In order to enable a person skilled in the art to understand the technical solutions of the present invention more clearly, the technical solutions of the present invention will be described in detail below with reference to specific examples.

### Example 1

20 g of phosphate buffer was left to stand at 4° C. for 10 min. 5 g of Pluronic F127 was added thereto and shaken for 1 min to disperse the Pluronic F127 in the phosphate buffer, and then left to stand at 4° C. for 3 days to fully dissolve the Pluronic F127. Finally, a Pluronic F127 solution with a concentration of 20 wt % was obtained as an inner material for coaxially printing.

0.0625 g of LAP photoinitiator was poured into a beaker. 16.1875 g of phosphate buffer and 3.75 g of polyethylene glycol diacrylate were added into the beaker and stirred magnetically in the dark at 40° C. in a rotational speed of 500 r/min for 20 min to fully dissolve the LAP photoinitiator and polyethylene glycol diacrylate. The mixture was transferred into a brown glass bottle and preserved at 4° C. for 20 min. 5 g of Pluronic F127 was added thereto and shaken for 1 min, and then left to stand at 4° C. for 3 days to fully dissolve the Pluronic F127. The obtained solution was used as an outer material for coaxially printing.

The inner and outer materials for coaxially printing were charged into a 5 mL syringe respectively and left to stand at 37° C. for 20 min. Parameters of a 3D printer were set as follows: 0.1 mm/min of inner extrusion speed, 0.3 mm/min of outer extrusion speed, and 700 mm/min of printing speed. The outer specification of a coaxial nozzle was 17 G, and the inner specification of the coaxial nozzle was 22 G. A high-temperature platform was selected as a receiving platform with a temperature set to 50° C. The three-dimensional scaffold has the layer height of 2 mm, the layer number of 3, the repeated printing number of 1, the spacing of 4×4 mm, the included angle of 90°, the length of 16 mm, the width of 16 mm, the brim width of 1 mm, and the brim speed of 500 mm/min. After coaxial printing was completed, the scaffold was irradiated with UV light (with a wavelength of 405 nm and a luminous intensity of 25 mW/cm²) for 10 min, so that the outer material underwent photocrosslinking, to finally form a three-dimensional hydrogel scaffold with stable structure. The hydrogel scaffold was then transferred into ultrapure water and incubated at 4° C. for 3 days to remove the thermosensitive material Pluronic F127 from both the inner and outer materials of the scaffold, to finally obtain a hollow hydrogel supporting scaffold shown in FIG. 2(a).

The hollow hydrogel supporting scaffold was soaked in 75% ethanol for sterilization for 3 h and soaked in PBS for washing for 12 h, and then soaked in replacing PBS for another 12 h, to remove the residual ethanol from the scaffold. Then, the scaffold was soaked in a complete medium and incubated in an incubator at 37° C. under 5% CO₂ for 1 day to obtain the scaffold shown in FIG. 2(b).

A cell-laden hydrogel matrix material was prepared. Gelatin methacrylate was selected as the matrix material. 0.75 g of LAP photoinitiator was added into 27.75 g of phosphate buffer, 1.5 g of GelMA material was added and stirred in a water bath at 40° C. in a rotational speed of 500 r/min for 30 min, and the mixture was filtered by a 0.45 μm filter to be sterilized. L929 fibroblasts were trypsinized and spin-centrifuged, and upon addition of the sterilized hydrogel material, were detached by pipetting to prepare the cell-laden hydrogel matrix with a density of 2×10⁶ cells/mL.

The hollow supporting scaffold was placed in a cell-culture dish, and the cell-culture dish was placed on ice. The cell-laden hydrogel matrix was poured on the hollow supporting scaffold for 1.5 mL per scaffold, and then irradiated with UV light (with a wavelength of 405 nm and a luminous intensity of 25 mW/cm²) for curing for 30 s, to obtain a vascular structure-containing large-scale biological tissue shown in FIG. 2(c).

In this example, the survival of cells in the biological tissue obtained in Example 1 was also observed through property testing:

Characterization of cell viability: Living/dead cells were stained with Calcein-AM/PI respectively, and the cell viability was characterized by using a fluorescence microscope. Calcein-AM can stain living cells with green fluorescence, while PI can stain dead cells with red fluorescence. FIG. 3 shows the staining of living and dead cells cultured for 1 day and 7 days in this example. FIG. 4(a) shows the viability of the cells cultured for 1 day and 7 days in Example 1. It can be seen that in the vascular structure-containing large-scale biological tissue prepared in this example, the cell viability is higher than 95% throughout the culture cycle and exceeds 99% on day 7.

Characterization of cell proliferation: The cell viability was characterized by CCK-8 assay. The scaffolds cultured for 1, 3, 5, and 7 days respectively were placed in a new 6-well plate and rinsed with PBS once. A complete medium and a CCK-8 solution were mixed in a volume ratio of 10:1 to prepare a working solution. The 6-well plate with each well containing 5 mL of the working solution was placed in an incubator at 37° C. under 5% CO₂ and incubated for 3 h. After the incubation was completed, the culture plate was taken out. The incubation solution was transferred from the 6-well plate to a 96-well plate for 100 μL per well, a total of 5 wells. The 96-well plate sub-loaded with the incubation solution was placed in a microplate reader to measure an absorbance at 450 nm. The proliferation of the cells cultured for 1, 3, 5, and 7 days is shown in FIG. 4(b). It can be learned that cells proliferate well in the vascular structure-containing large-scale biological tissue.

### Example 2

To verify that the biological tissue provided by the present invention can adjust the porosity of the duct walls of the supporting scaffold structure by changing the concentration of the crosslinking material in the coaxially printing outer material, this example describes the method for adjusting the porosity. The crosslinking material is polyethylene glycol diacrylate.

20 g of phosphate buffer was left to stand at 4° C. for 10 min. 5 g of Pluronic F127 was added thereto and shaken for 1 min to disperse the Pluronic F127 in the phosphate buffer, and then left to stand at 4° C. for 3 days to fully dissolve the Pluronic F127. Finally, a Pluronic F127 solution with a concentration of 20 wt % was obtained as an inner material for coaxially printing.

A LAP photoinitiator was poured into a beaker. A phosphate buffer and polyethylene glycol diacrylate were added into the beaker and stirred magnetically in the dark at 40° C. in a rotational speed of 500 r/min for 20 min to fully dissolve the LAP photoinitiator and polyethylene glycol diacrylate. The mixture was transferred into a brown glass bottle and preserved at 4° C. for 20 min. Pluronic F127 was added thereto and shaken for 1 min, and then left to stand at 4° C. for 3 days to fully dissolve the Pluronic F127. The finally obtained solution contained 20 wt % of Pluronic F127 and 10 wt %, 15 wt %, or 20 wt % of polyethylene glycol diacrylate marked as P10, P15, or P20 respectively, which was used as an outer material for coaxially printing.

The inner and outer materials for coaxially printing were charged into a 5 mL syringe respectively and left to stand at 37° C. for 20 min. Parameters of a 3D printer were set as follows: 0.1 mm/min of inner extrusion speed, 0.3 mm/min of outer extrusion speed, and 700 mm/min of printing speed. The outer specification of a coaxial nozzle was 17 G, and the inner specification of the coaxial nozzle was 22 G. A high-temperature platform was selected as a receiving platform with a temperature set to 50° C. The three-dimensional scaffold has the layer height of 2 mm, the layer number of 1, the repeated printing number of 1, the spacing of 4×4 mm, the included angle of 90°, the length of 40 mm, the width of 25 mm, the brim width of 1 mm, and the brim speed of 500 mm/min. After coaxial printing was completed, the scaffold was irradiated with UV light (with a wavelength of 405 nm and a luminous intensity of 25 mW/cm²) for 10 min, so that the outer material underwent photocrosslinking, to finally form a three-dimensional hydrogel scaffold with stable structure. The hydrogel scaffold was prepared into U-shaped structures, then transferred into ultrapure water and soaked at 4° C. for 1 day and 3 days respectively, to remove the thermosensitive material Pluronic F127 from both the inner and outer materials of the scaffold.

After having been soaked, the scaffold prepared from the solution containing 10 wt % of polyethylene glycol diacrylate failed to form, so the scaffolds prepared from the solutions containing 15 wt % and 20 wt % of polyethylene glycol diacrylate were used for subsequent testing.

The U-shaped structures soaked for 1 day and 3 days respectively were taken out, with those prepared from the solution containing 15 wt % of polyethylene glycol diacrylate being marked as P15-1 and P15-3 respectively and those prepared from the solution containing 20 wt % of polyethylene glycol diacrylate being marked as P20-1 and P20-3 respectively. Subsequently, the diffusion test was carried out to evaluate the regulation effect of the concentration of the crosslinking material in the outer material on the porosity of the duct walls according to the diffusion of small molecules from the inside to the outside of the ducts. During the diffusion test, 30 μL of 0.5 wt % small-molecule eosin Y dye was injected into the U-shaped structure, which was put into an EP tube. 800 μL of PBS was added into the EP tube and left in a 37° C. environment. The PBS was sampled at 30 min, 1 h, 3 h, 6 h, 12 h, and 24 h from the EP tube, respectively. 300 μL of PBS was pipetted out of the EP tube for each sample and transferred into a 96-well plate for 100 μL per well. That is, the PBS pipetted out for each sample was injected into 3 wells. An absorbance was measured at a wavelength of 525 nm by a microplate reader.

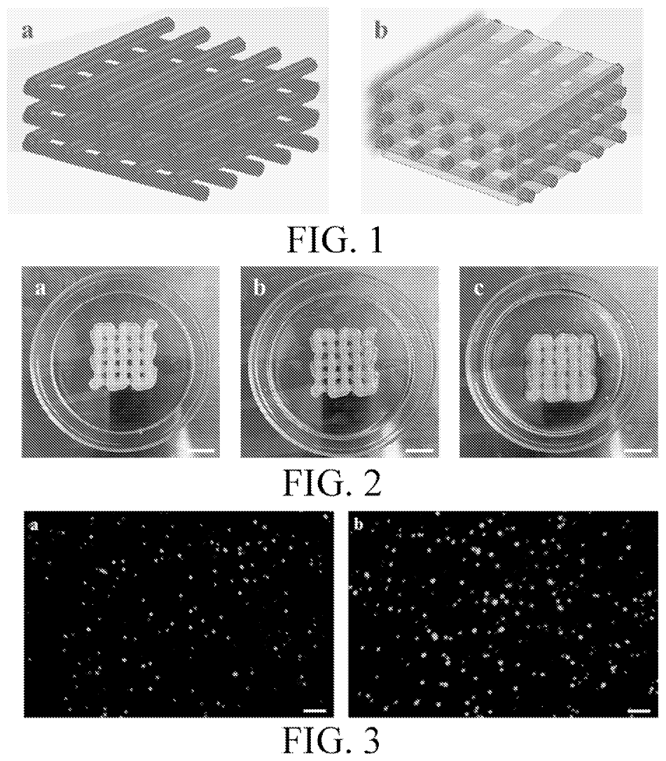

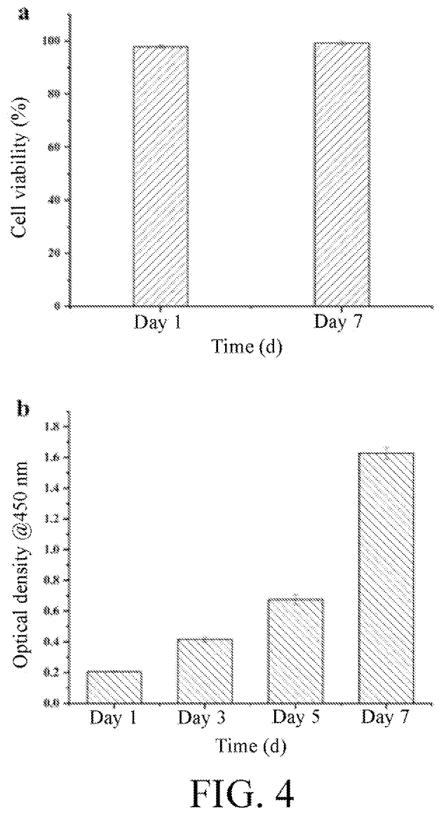

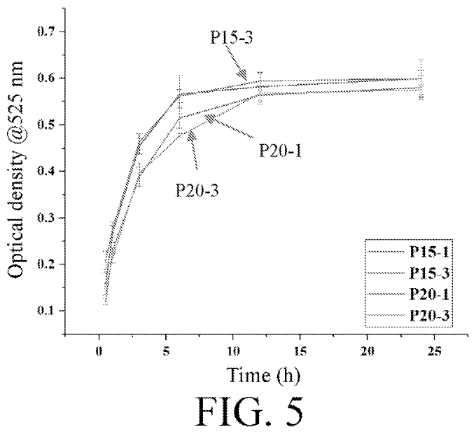

FIG. 5 shows the diffusion of small molecules in the hydrogel ducts. It can be learned from the figure that for the two coaxially printing outer materials containing 15 wt % and 20 wt % of polyethylene glycol diacrylate respectively, there is no significant difference in the diffusion behavior between the obtained hydrogel ducts soaked for 1 day and 3 days, indicating that the thermosensitive material is basically removed after being soaked for 1 day. At all time points, the absorbance of the solution outside the hydrogel ducts prepared from the material containing 15 wt % of polyethylene glycol diacrylate is higher than that from the material containing 20 wt % of polyethylene glycol diacrylate, indicating that the porosity of the hydrogel ducts prepared from the material containing 15 wt % of polyethylene glycol diacrylate is higher than that from the material containing 20 wt % of polyethylene glycol diacrylate. Therefore, the porosity of the duct walls of the supporting scaffold structure can be adjusted by changing the concentration of the crosslinking material in the coaxially printing outer material.

The foregoing descriptions are merely preferred examples of the present invention and are not intended to limit the present invention. A person skilled in the art may make various alterations and variations to the present invention. Any modification, equivalent replacement, or improvement made within the spirit and principle of the present invention shall fall within the protection scope of the present invention.

1. A vascular structure-containing large-scale biological tissue, the biological tissue comprising a cell-laden hydrogel matrix and a supporting scaffold, wherein the supporting scaffold is a hollow duct inserted into the cell-laden hydrogel matrix to mimic vascular structures and contains a thermosensitive material.

2. The vascular structure-containing large-scale biological tissue according to claim 1, wherein the supporting scaffold is one or more ducts connected or not connected to each other; and
further, the supporting scaffold has multiple layers of ducts superposed in the cell-laden hydrogel matrix, and the ducts of adjacent layers are arranged crosswise or vertically.

3. A method for constructing the vascular structure-containing large-scale biological tissue according to claim 1, the method comprising: coaxially printing inner and outer materials on a receiving platform to form a scaffold structure, the outer material being a composite material of a thermosensitive material and a crosslinking material, and the inner material being a thermosensitive material; curing the outer material of the scaffold structure after the printing to obtain a stable scaffold structure; incubating the stable scaffold structure at a melting temperature of the thermosensitive material to melt the thermosensitive material of the inner and outer materials to obtain a hollow supporting scaffold; and pouring a cell-laden hydrogel matrix into the supporting scaffold and curing it.

4. The method for constructing the vascular structure-containing large-scale biological tissue according to claim 3, wherein the coaxially printing inner and outer materials on a receiving platform to form a scaffold structure comprises:
preparing the inner and outer materials for coaxially printing, the inner material being the thermosensitive material, and the outer material being a composite material of the thermosensitive material and the crosslinking material, charging the liquid inner and outer materials into syringes separately, and incubating them at a first temperature to form a gel;
loading the syringes containing the gel-like inner and outer materials into an extrusion 3D printer, connecting a nozzle for coaxial printing to the syringe, setting process parameters of the 3D printer, activating the receiving platform of the 3D printer, and setting a temperature of the receiving platform; and
starting software of the 3D printer to enable the nozzle for coaxial printing to move according to a predetermined trajectory and extrude the gel materials from the syringe on the receiving platform to form the scaffold structure, and processing the scaffold structure to cure the crosslinking material in the outer composite material, to obtain the stable scaffold structure.

5. The method for constructing the vascular structure-containing large-scale biological tissue according to claim 4, wherein in the printing method, the inner thermosensitive material is Pluronic F127 or its derivatives or gelatin; further, the inner thermosensitive material is Pluronic F127;
or the crosslinking material is polyethylene glycol diacrylate, gelatin methacrylate, sodium alginate, or a mixture thereof.

6. The method for constructing the vascular structure-containing large-scale biological tissue according to claim 5, wherein the inner material is formulated by dissolving Pluronic F127 in a phosphate solution at a mass fraction of 10-30%; and
the outer material is formulated by fully dissolving a photoinitiator and polyethylene glycol diacrylate in a phosphate solution, prior to addition of Pluronic F127 for dissolving, wherein Pluronic F127 has a mass concentration of 10-30% and polyethylene glycol diacrylate has a mass concentration of 5-20%.

7. The method for constructing the vascular structure-containing large-scale biological tissue according to claim 4, wherein the first temperature is 4-40° C., and the incubation time is 10-30 min;
or the outer specification of the nozzle for coaxial printing is 14-21 G, and the inner specification of the nozzle for coaxial printing is 18-30 G;
or during 3D printing, a moving speed of the nozzle for coaxial printing is 400-800 mm/min, and an inner-to-outer extrusion speed ratio is 1:4-1:1;
or the receiving platform is a high-temperature platform or a low-temperature platform with a temperature set to 4-50° C.; in a specific implementation, the receiving platform is a high-temperature platform with a temperature set to 50° C.

8. The method for constructing the vascular structure-containing large-scale biological tissue according to claim 3, wherein during the construction of the hollow supporting scaffold, the stable scaffold structure is soaked in a phosphate buffer or ultrapure water for incubation for 1-5 days, with a melting temperature of the thermosensitive material being 4-40° C.

9. The method for constructing the vascular structure-containing large-scale biological tissue according to claim 8, wherein the stable scaffold structure is soaked in ultrapure water for incubation at a temperature of 4° C. for 1-3 days;
further, after the hollow supporting scaffold is constructed, the method also comprises a step for sterilization by soaking the hollow supporting scaffold in a 75% ethanol solution for 1-5 h.

10. The method for constructing the vascular structure-containing large-scale biological tissue according to claim 3, wherein the cell-laden hydrogel matrix material comprises but is not limited to collagen, hyaluronic acid, sodium alginate, gelatin methacrylate, or a combination thereof, with a mass concentration of 5-15%.

11. A method for constructing the vascular structure-containing large-scale biological tissue according to claim 2, the method comprising: coaxially printing inner and outer materials on a receiving platform to form a scaffold structure, the outer material being a composite material of a thermosensitive material and a crosslinking material, and the inner material being a thermosensitive material; curing the outer material of the scaffold structure after the printing to obtain a stable scaffold structure; incubating the stable scaffold structure at a melting temperature of the thermosensitive material to melt the thermosensitive material of the inner and outer materials to obtain a hollow supporting scaffold; and pouring a cell-laden hydrogel matrix into the supporting scaffold and curing it.

12. The method for constructing the vascular structure-containing large-scale biological tissue according to claim 11, wherein the coaxially printing inner and outer materials on a receiving platform to form a scaffold structure comprises:
preparing the inner and outer materials for coaxially printing, the inner material being the thermosensitive material, and the outer material being a composite material of the thermosensitive material and the crosslinking material, charging the liquid inner and outer materials into syringes separately, and incubating them at a first temperature to form a gel;
loading the syringes containing the gel-like inner and outer materials into an extrusion 3D printer, connecting a nozzle for coaxial printing to the syringe, setting process parameters of the 3D printer, activating the receiving platform of the 3D printer, and setting a temperature of the receiving platform; and
starting software of the 3D printer to enable the nozzle for coaxial printing to move according to a predetermined trajectory and extrude the gel materials from the syringe on the receiving platform to form the scaffold structure, and processing the scaffold structure to cure the crosslinking material in the outer composite material, to obtain the stable scaffold structure.

13. The method for constructing the vascular structure-containing large-scale biological tissue according to claim 12, wherein in the printing method, the inner thermosensitive material is Pluronic F127 or its derivatives or gelatin; further, the inner thermosensitive material is Pluronic F127;
or the crosslinking material is polyethylene glycol diacrylate, gelatin methacrylate, sodium alginate, or a mixture thereof.

14. The method for constructing the vascular structure-containing large-scale biological tissue according to claim 13, wherein the inner material is formulated by dissolving Pluronic F127 in a phosphate solution at a mass fraction of 10-30%; and
the outer material is formulated by fully dissolving a photoinitiator and polyethylene glycol diacrylate in a phosphate solution, prior to addition of Pluronic F127 for dissolving, wherein Pluronic F127 has a mass concentration of 10-30% and polyethylene glycol diacrylate has a mass concentration of 5-20%.

15. The method for constructing the vascular structure-containing large-scale biological tissue according to claim 12, wherein the first temperature is 4-40° C., and the incubation time is 10-30 min;
or the outer specification of the nozzle for coaxial printing is 14-21 G, and the inner specification of the nozzle for coaxial printing is 18-30 G;
or during 3D printing, a moving speed of the nozzle for coaxial printing is 400-800 mm/min, and an inner-to-outer extrusion speed ratio is 1:4-1:1;
or the receiving platform is a high-temperature platform or a low-temperature platform with a temperature set to 4-50° C.; in a specific implementation, the receiving platform is a high-temperature platform with a temperature set to 50° C.

16. The method for constructing the vascular structure-containing large-scale biological tissue according to claim 11, wherein during the construction of the hollow supporting scaffold, the stable scaffold structure is soaked in a phosphate buffer or ultrapure water for incubation for 1-5 days, with a melting temperature of the thermosensitive material being 4-40° C.

17. The method for constructing the vascular structure-containing large-scale biological tissue according to claim 16, wherein the stable scaffold structure is soaked in ultrapure water for incubation at a temperature of 4° C. for 1-3 days;
further, after the hollow supporting scaffold is constructed, the method also comprises a step for sterilization by soaking the hollow supporting scaffold in a 75% ethanol solution for 1-5 h.

18. The method for constructing the vascular structure-containing large-scale biological tissue according to claim 11, wherein the cell-laden hydrogel matrix material comprises but is not limited to collagen, hyaluronic acid, sodium alginate, gelatin methacrylate, or a combination thereof, with a mass concentration of 5-15%.
